(19) United States
(12) Patent Application Publication
(10) Pub. No.: US 2009/0093887 A1
(43) Pub. Date: Apr. 9, 2009

(54) Implantable Joint Prosthesis

(75) Inventors: William Lindsay Walter, New South Wales (AU); Ronald Mark Gillies, New South Wales (AU); Shane Donohoo, New South Wales (AU)

Correspondence Address: SCHWEGMAN, LUNDBERG & WOESSNER, P.A., P.O. BOX 2938, MINNEAPOLIS, MN, 55402, US

(73) Assignee: Silesco Pty Ltd., Waverton, NSW (AU)

(21) Appl. No.: 12/087,503

(22) PCT Filed: Dec. 22, 2006
(86) PCT No.: PCT/AU2006/001968

§ 371 Date: Dec. 19, 2008

(30) Foreign Application Priority Data
Jan. 9, 2006 (AU) 2006900086
Jan. 27, 2006 (AU) 2006900406
Mar. 15, 2006 (AU) 2006901324
Aug. 10, 2006 (AU) 2006904349

(51) Int. Cl. A61F 2/32; A61F 2/30

(52) U.S. Cl. 623/22.11; 623/18.11; 623/22.4

(57) ABSTRACT

An implantable joint prosthesis configured such that it does not squeak during movements of a subject. The joint prosthesis includes a means to modify the dynamic response of parts of the prosthesis such the response is not audible to the human ear.

### CROSS-REFERENCE TO RELATED APPLICATIONS

The present application claims priority from Australian Provisional Patent Application No 2006900086, Australian Provisional Patent Application No 2006900406, Australian Provisional Patent Application No 2006901324, Australian Provisional Patent Application No 2006904349 filed on 9 Jan. 2006, 27 Jan. 2006, 15 Mar. 2006, 19 Aug. 2006, the contents of which are incorporated herein by reference.

### FIELD OF THE INVENTION

The present invention relates to a joint replacement prosthesis for implantation into the body of an individual, in particular to a joint replacement prosthesis which can function to replace at least a part of the joint of an individual and which can operate in a substantially quiet mode.

### BACKGROUND ART

Joints, such as the hip, knee, ankle, elbow and shoulder, are formed by the ends of two or more bones connected by cartilage tissue, which in healthy joints, acts as a protective cushion for the joint, allowing smooth, low friction movement of the joint. Through disease, injury or old age, the cartilage may become damaged causing the tissue around the joint to become inflamed and hence cause pain to the individual, which over time, can cause the cartilage to erode, thereby resulting in the rough edges of the bone contacting and rubbing against each other causing further damage to the joint and significant pain to the individual.

If only some of the joint is damaged, it may be possible for a surgeon to repair or replace the damaged portions of the joint through a variety of surgical procedures and treatments. However, if the entire joint becomes damaged or deteriorates significantly through such conditions as osteoarthritis, rheumatoid arthritis, or avascular necrosis, reparative treatment may not be possible and a more radical treatment may be necessary.

Such conditions have been successfully treated by total replacement joints to replace the diseased or damaged joint. To replace the knee or hip joint, a surgeon typically removes the diseased or damaged parts and inserts artificial parts, commonly referred to as prostheses or implants. As artificial joints and the surgical techniques to implant them have evolved over time, such joint replacements are becoming more accessible to a number of individuals and function more like a healthy natural joint.

In this regard, in recent times hip replacements have been among the most commonly performed orthopedic procedures and have been shown to be a successful means for relieving pain and restoring mobility to the individual. In a total hip replacement procedure, the ball part of the joint is removed and replaced with a ball attached to a stem which is wedged into a hollowed out space formed in the femur of the individual. Damaged bone and cartilage are removed from the socket and a cup-like component is inserted into the socket to receive the ball of the stem.

An alternative to total hip replacement is a procedure referred to as hip resurfacing, which has also been successful in treating damaged hip joints. In such a procedure, rather than replacing the femoral head, the head of femur is preserved and reshaped and the reshaped bone is then capped with a metal ball that is fixedly attached to the neck of the femur. The socket/acetabulum is prepared in a similar manner to a total hip replacement to receive the metal ball of the femur. As is appreciated, such a procedure requires less bone removal than a total hip replacement, however relies upon the same principles to replicate the action of a healthy hip joint.

Due to the success of hip replacement surgery, the procedure is being performed in patients of various ages and as such, it is important that the implants last the lifetime of the recipient, which can be as long as 70-80 years for some recipients. For this reason, various types of hip replacement implants have been proposed using a variety of different materials. There are implants whereby the ball is made from a hard material (such as metal or ceramic) and the cup is made from a plastic (typically polyethylene) which may or may not have a metal backing of titanium, stainless steel or cobalt chrome. Such implants tend to wear over time as the plastic material wears out. This can be at a rate of 0.1 mm per year or more. To avoid this, alternative bearing surfaces to the hard material-on-plastic have been proposed, which are referred to as hard-on-hard. These systems typically employ metal-on-metal or ceramic-on-ceramic bearing surfaces, whereby substantially all parts of the prosthesis are made from a metal or ceramic material. Such hard-on-hard systems have been shown to significantly reduce the amount of wear and have the potential to increase the life of the implant.

Whilst hip replacement surgery has been proven relatively successful in restoring movement to individuals and relieving pain previously experienced in the joint, in some instances an undesirable outcome has been the presence of an audible squeak associated with the implant in some individuals. Such a squeak may be experienced by the individual when bending or during walking and can be a source of embarrassment and distress to the individual. Implant squeaking is more prevalent in implants with hard-on-hard bearing surfaces (metal-on-metal implants or ceramic-on-ceramic implants).

In the case of modular acetabular components where there is a ceramic insert and a titanium shell the two components are joined by a locking mechanism in the form of a taper. This locking mechanism is designed for generally axial loading. While such a locking mechanism may perform adequately under generally axial loads they do not perform well under other loadings and in particular edge loading. Edge loading produces loads that are nearly perpendicular to the axis of the component. Under these conditions the prior art inserts can tilt out of the shell, uncoupling the two components. Such uncoupling may lead to undesirable squeaking of the prosthesis.

Therefore, there is a need to provide a joint replacement method and prosthesis that can be employed to relieve pain associated with the damaged joint and to restore mobility to the joint, whilst reducing the occurrence of audible squeaking associated with the prosthesis.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In a first aspect, the present invention is an implantable joint prosthesis comprising:

a first component attachable to a first bone of a recipient; and

a second component attachable to a second bone of a recipient,

wherein said first and second components are arranged to facilitate relative movement between said first and second bone of the recipient, and at least one of said first and/or second components comprises at least one modifying means, said modifying means modifying the first and/or second component such that a dynamic response of at least a part of the first and/or second component to a stimulus is modified.

In a second aspect, the present invention is an acetabular component of an implantable hip prosthesis, said acetabular component having a main axis and comprising a cup member shaped to receive an insert member substantially therein, wherein the insert member and the cup member are coupled together by a primary locking mechanism, said primary locking mechanism retaining the insert member and the cup member in coupling engagement when said acetabular component is subjected to a load substantially along said main axis;

the prosthesis characterised in that the acetabular component comprises a secondary locking mechanism to couple together the insert member and the cup member.

Typically, said secondary locking mechanism retains the insert member and the cup member in coupling engagement when said acetabular component is subjected to a load that deviates from along said main axis.

In the second aspect, the secondary locking mechanism may retain the locking member and the cup member in locking engagement when the load applied to the acetabular component is at an angle to the main axis. The angle of the load may be from 1° to 90° relative to the main axis. The angle may be between 10° and 70° relative to the main axis. Furthermore, the angle of load may be between 20° and 50° relative to the main axis.

The dynamic response may comprise the resonant frequency of at least a part of the first and/or second components.

In one embodiment, the magnitude of the dynamic response may be modified such that any noise resulting from a resonance of said at least a part of the first and/or second component is reduced and preferably to a level that is not audible to a human.

Alternatively, the frequency of the dynamic response may be modified. The frequency of the dynamic response may be modified to a frequency greater than 7 KHz. Preferably, the frequency of the dynamic response is modified to a frequency greater than 10 KHz. Still further, the frequency of the dynamic response may be modified to a frequency in the range of 10 KHz to 20 KHz. Yet further, the dynamic response frequency may be modified to a frequency greater than 20 KHz. The frequency of the dynamic response may also be modified to a frequency less than 1 KHz and preferably less than 500 Hz; more preferably less than 20 Hz.

In a further embodiment, both the magnitude and the particular frequency of the dynamic response may be modified.

The implantable joint prosthesis may comprise an implantable hip prosthesis. The implantable hip prosthesis may be a total hip prosthesis or a partial hip prosthesis.

The first component may comprise a femoral component for attachment to the femur of the recipient. The femoral component may be in the form of a stem which is insertable into a cavity formed in the femoral bone. The stem may include a neck region which projects from the femur. The femoral component may also comprise a head element arranged to be received by the neck region of the stem. The head element may be in the form of a ball or part thereof. The surface of the ball may be substantially spherical in configuration and may be made from a hard material, such as a ceramic or a metal.

The second component may be an acetabular component for attachment to the acetabulum of the pelvis. The acetabular component may comprise a cup which is configured to be anchored into the acetabulum. The cup may receive the head element of the femoral component and is shaped to substantially conform to the head element. In one form, the cup may comprise an insert which is configured to be received within the cup. The insert may be made from a hard material such as a ceramic or a metal. In this arrangement, the insert may receive the head element of the femoral component to facilitate articular movement between the femoral component and the acetabular component.

The insert may include a main body having an upper face comprising a rim and a recessed inner surface. The recessed inner surface may receive the femoral component. An outer surface of the insert may comprise a tapered region that extends from the rim towards a base of the insert.

Similarly, the cup may comprise an upper face having a rim and a recessed inner surface. The recessed inner surface may receive the insert. A region of the inner surface of the cup may be tapered. The tapered region of the insert and the tapered region of the cup may be wholly or partially engageable with each other. The region of engagement between the tapered surfaces provides an interface between the cup and the insert.

The modifying means of the present invention may comprise a number of means with the common feature being that it alters the dynamic response of at least a part of the prosthesis by either modifying the magnitude of the response or modifying the actual frequency of the response.

The modifying means may modify the physical properties of the first and/or second component or parts thereof. Modifications of various physical properties may change the dynamic response of the first or the second component or parts thereof such that the response is not audible to humans. Examples of modifying means that modify the physical properties of the components or parts thereof are discussed in further detail below and include but are not limited to shape modifying members, stiffness modifying members and mass modifying members of the first and/or second component or parts thereof.

In a further embodiment, the first and/or second component may be configured such that the frequency of the dynamic response is damped. In this embodiment, the first and/or second component may comprise a damping member to dampen certain frequencies such that the dynamic response is shifted out of an audible range.

The acetabular cup of the acetabular component may comprise at least one shape modifying member. Preferably, the cup includes a plurality of shape modifying members. The shape modifying members are typically ribs or struts that extend outwardly from an outer surface of the cup. The ribs or struts may extend substantially around the circumference of the cup, either longitudinally or laterally relative to the main axis of the cup. The ribs or struts may be evenly spaced. Preferably, the ribs or struts may be asymmetrically spaced.

In a further embodiment, the acetabular component comprises at least one stiffness modifying member. Preferably, the stiffness modifying member increases the stiffness of the acetabular cup such that it is less likely to distort. Typically, the stiffness modifying member increases either or both the hoop stiffness and the bending stiffness of the acetabular cup.

In many hip prostheses, the acetabular cup is relatively flexible. An insert is fitted within the cup and a femoral head received in the insert. When subjected to various loads, the relatively flexible cup may undergo a distortion. The relationship between the insert and the cup, therefore, changes. For example, the insert may become uncoupled from the cup as will be discussed in further detail below This uncoupling may allow the cup to resonate at a particularly frequency that is audible to a human being.

The stiffness modifying member may comprise one or more ribs or struts positioned on the outer surface of the cup to increase the stiffness of the cup. The ribs or struts may be the same as the shape modifying members discussed above and it should be appreciated that the effects of the modifying means may overlap; a member that alters the shape of a component may also modify the stiffness and vice versa.

The stiffness modifying member may further comprise a ring member that is stiffer than the cup. The ring member may be made from the same material as the material of the cup. In this embodiment, the ring member may include stiffening features to increase the stiffness of the ring member. For example, the stiffening features may include ribs or struts that extend outwardly from the ring member. Alternatively, the ring member may include a flange member that extends outwardly therefrom.

The cup may be made from titanium and the ring member may be made from a different material selected from cobalt chrome alloy and stainless steel.

The ring member may extend substantially circumferentially around the outer surface of the cup. The ring member may extend around the entire circumference of the acetabular cup. The ring member may be substantially flush with the rim of the cup. Alternatively, the ring member may extend beyond the rim of the cup or may be recessed relative to the rim of the cup. The ring member may be bonded to the cup. For example, the ring member may be press fitted to the outer surface of the cup. Alternatively, the ring member may be bonded to the acetabular cup by hot isostatic pressing (HIPing).

The stiffness of the cup may also be modified by altering the thickness of the cup. Particularly, the diameter of at least a portion of the tapered region of the cup may be varied in the range of approximately between 2 mm to 10 mm

Resonance of the cup in an audible range may also be prevented by locking the insert and the cup together under all loading conditions (particularly under loading of the femoral head on the edge of the insert) such that the cup is not free to resonate at its audible natural frequency ie the insert and the cup act as a composite structure with a different resonant frequency to that of the cup alone. The modifying means of the first aspect or the secondary locking mechanism of the second aspect may cause the insert and the cup to remain in locking engagement under all loading conditions.

The complementary tapers of the insert and the cup may allow for a press fit between the two components to friction fit them together. While the taper provides a sufficient lock under generally axial loads, it may not sufficiently lock the two components together under other loads that deviate from the main axis of the acetabular component, including during edge loading and impingement (wherein the neck of the femoral component hits the edge of the acetabular component).

Preferably, the insert and the cup are locked together using either mechanical details or by altering other variables including friction, taper angle of the insert and the stiffness of the cup as will be discussed in more detail below.

In one embodiment of the second aspect of the invention, the secondary locking mechanism comprises at least one mechanical locking mechanism to secure the insert within the cup such that the two components act as a composite structure under clinically relevant loads and particularly during edge loading and impingement. The secondary locking mechanism of this embodiment may comprise a mechanical detail on the insert and a complementary receiving member on the cup. Further, the insert may include an intermediate member positioned substantially around the circumference of the insert. In this embodiment, the mechanical detail may be positioned on the intermediate member rather than on the insert.

In a further embodiment of the second aspect, the secondary locking mechanism may comprise the stiffness of the cup. In this embodiment, the cup may include a ring member as described above. Alternatively, or in addition to the ring member, the stiffness of the acetabular cup may be altered by altering the thickness of at least a portion of the tapered region of the cup. Particularly, the thickness of the entire region of the cup that forms an interface with the insert may be varied. The thickness may be varied in the range from 2 mm to approximately 10 mm. Preferably, the range of thickness variation is between 2 mm and 5 mm. Still further, the thickness variation may be in the range of 2 mm to 2.85 mm.

Further, the stiffening features may comprise ribs, struts or flanges as discussed above.

The secondary locking mechanism may comprise a combination of two or more of said ring member, the thickness of the cup and stiffening features. The locking mechanism of this embodiment is achieved by providing an optimal stiffness of the cup such that the cup will not be distorted under load such that the insert is uncoupled from the cup.

The secondary locking mechanism may further comprise a combination of cup stiffness and the friction coefficient between the insert and the cup. If the friction coefficient is low, the stiffness of the cup may be increased to allow the insert and the cup to act as a composite structure.

Additionally, the secondary locking mechanism may comprise a combination of optimal stiffness and taper angle of the insert and the cup. As the taper angle decreases relative to the main axis of the acetabular component, the axial capacity (ability for the insert and the cup to remain as a composite under axial load) of the acetabular component may decrease but the edge loading capacity (ability of the insert and the cup to remain as a composite when the load deviates from the main axis) may increase. The decrease in axial capacity in this embodiment may be countered by the stiffness of the cup, and particularly by an increase in stiffness in accordance with the various embodiments described herein. Alternatively, a base region of the insert and a base region of the cup may be engageable with each other to provide an additional load path to counter the decrease in axial capacity.

Preferably, the taper angle is in the range of between 4 degrees and 10 degrees relative to the main axis of the cup.

Still further, the secondary locking mechanism may comprise a combination of stiffness of the cup, friction coefficient between cup and insert and taper angles of the insert and the cup.

The secondary locking mechanism preferably minimises the motion of the insert and the cup relative to each other. Preferably, the relative motion between the insert and the cup is less than 40 microns when the acetabular component is subjected to a load that deviates from the main axis of the component.

The femoral component of the prosthesis may also comprise the modifying means. The modifying means may modify the geometric shape of the femoral component. Still further, the modifying means may modify the stiffness of the femoral component. The modifying means may also modify the mass distribution of the femoral component. The shape and/or stiffness and/or mass distribution of either or both of the femoral stem and the bead element of the femoral component may be modified by the modifying means.

In another embodiment, the geometric structure of the first and/or second components are modified such that the resonant frequency of the first component is mismatched with the resonant frequency of the second component to reduce the tendency for mode coupling between the two components.

### BRIEF DESCRIPTION OF THE DRAWINGS

By way of example only, the invention is now described with reference to the accompanying drawings:

FIG. 1 is a view of a normal human hip joint, showing the femur in position with respect to the pelvis;

FIG. 2A is an exploded view of one example of a total hip prosthesis suitable for use with the present invention;

FIG. 2B is an exploded view of a hip resurfacing prosthesis suitable for use with the present invention;

FIG. 3 is a diagrammatical view of the total hip prosthesis of FIG. 2A following implantation;

FIG. 4 is a schematic partial cross-sectional view of an acetabular cup in accordance with one embodiment of the present invention;

FIG. 5 is a schematic view of an acetabular cup in accordance with another embodiment of the present invention;

FIG. 6 is a schematic view of an acetabular cup in accordance with yet another embodiment of the present invention;

FIG. 7 is a schematic view of an acetabular cup in accordance with yet another embodiment of the present invention;

FIG. 8 is a schematic view of an acetabular cup in accordance with yet another embodiment of the present invention;

FIG. 9 is a schematic partial cross-sectional view of an acetabular cup in accordance with yet another embodiment of the present invention;

FIG. 10 is a schematic partial cross-sectional view of an acetabular cup in accordance with yet another embodiment of the present invention;

FIG. 11 is a schematic partial cross-sectional view of an acetabular cup in accordance with another embodiment of the present invention;

FIG. 12 is a schematic partial cross-sectional view of an acetabular cup in accordance with another embodiment of the present invention;

FIG. 13 is an exploded view of an insert and cup arrangement in accordance with an embodiment of the present invention;

FIG. 14 is a view of a femoral stem component of a prosthesis employing a damping spacer in accordance with an embodiment of the present invention;

FIG. 15 is an alternative view of the femoral stem component employing the damping spacer of FIG. 14;

FIG. 16 is another alternative view of the femoral stem component employing the damping spacer of FIG. 14;

FIG. 17 is a view of a femoral stem component of a hip prosthesis employing a mass damper in accordance with an embodiment of the present invention;

FIG. 18 is a view of a femoral stem component of a hip prosthesis employing a liquid mass damper in accordance with an embodiment of the present invention;

FIG. 19 is a front and side view of a femoral head portion of a hip prosthesis employing a damping spacer in accordance with an embodiment of the present invention;

FIG. 20 is a front and side view of an acetabular cup portion of a hip prosthesis in accordance with an embodiment of the present invention;

FIG. 21 is a front and end view of a femoral stem component of a hip prosthesis in accordance with an embodiment of the present invention;

FIG. 22 is a view of a femoral stem component of a hip prosthesis in accordance with an embodiment of the present invention;

FIG. 23 is a view of an acetabular cup portion of a hip prosthesis in accordance with an embodiment of the present invention;

FIG. 24 is a cross sectional view of the acetabular portion of another embodiment of the present invention;

FIGS. 25A and 25B are side views of the damping device of FIG. 29 in a relaxed and compressed state respectively;

FIG. 26 shows a top view of an alternative damping device for use with the embodiment as shown in FIG. 24;

FIG. 27 shows side, top and enlarged side views of yet another alternative damping device for use with the embodiment as shown in FIG. 24;

FIG. 28 shows yet another embodiment of a damping system in accordance with another embodiment of the present invention;

FIG. 29 shows a cross-sectional side view of the acetabular portion of another embodiment of the present invention;

FIG. 30 shows a cross sectional side view of a stabilising plate in accordance with another embodiment of the present invention being employed to restrict unwanted movement between the cup and insert of the acetabular portion of the prosthesis;

FIGS. 31A and 31B show side and top views respectively of one embodiment of the stabilising plate of FIG. 30;

FIGS. 32A and 32B show side and top views respectively of another embodiment of the stabilising plate of FIG. 30;

FIGS. 33 to 36 show further alternative embodiments of the stabilising plate of FIG. 35;

FIGS. 37 and 38 show an arrangement for locating the insert and cup of the acetabular portion of the prosthesis in position in accordance with yet another embodiment of the present invention.

FIGS. 39a to 39e depict embodiments of locking mechanisms of the present invention;

FIG. 40a is a cross-sectional view of an acetabular component of the invention showing a locking mechanism;

FIG. 40b is a top plan view of the acetabular component of FIG. 40a;

FIG. 41 a is a partial cross-sectional view of the acetabular component showing a locking mechanism of a further embodiment of the invention;

FIG. 42 is a graph depicting the inter-relationship between properties of components of the prosthesis of the present invention;

FIG. 43 is a partial cross-sectional view of a further embodiment of the present invention.

FIG. 44 is a table showing the frequency of various components of a prosthesis;

FIG. 45 is a graph showing the relationship between friction coefficient and stiffness of components of the prosthesis; and

FIGS. 46a and 46b show a further embodiment of a component of the prosthesis of the present invention.

### DETAILED DESCRIPTION OF AN EXEMPLARY EMBODIMENT OF THE PRESENT INVENTION

The present invention will be described in relation to a hip joint prosthesis, however it will be appreciated by a person skilled in the art that the present invention could be equally be applied to a prosthesis suitable for use with any joint, whether the prosthesis be a partial or full replacement of the natural joint.

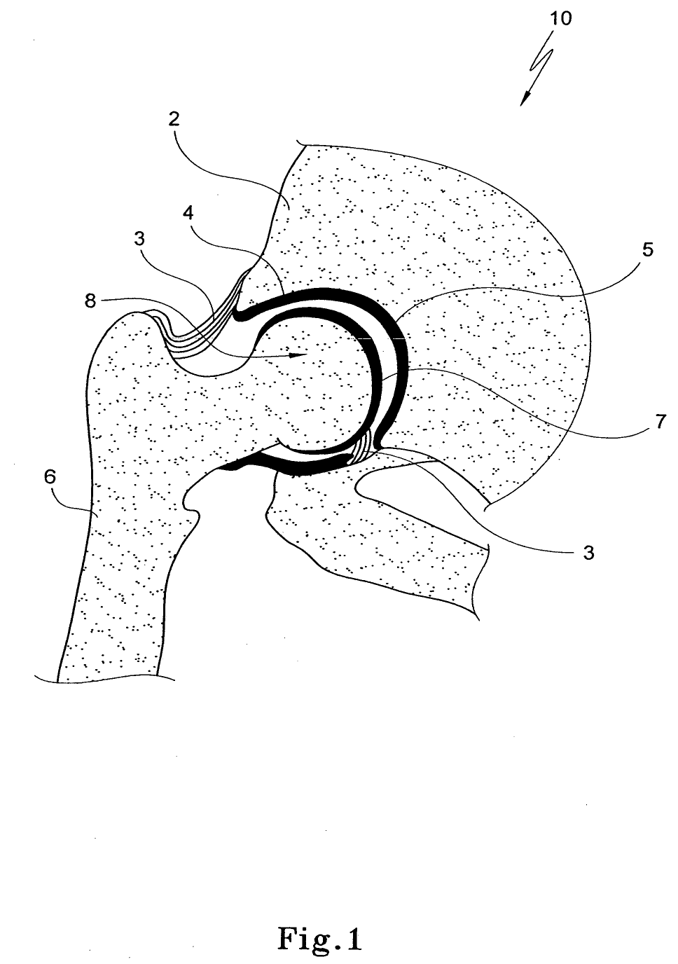

With regard to FIG. 1, a part of a normal hip joint 10 is shown. The hip joint 10 generally functions to connect the legs to the torso of an individual, and hence comprises the pelvis 2 having the acetabulum (or socket) 4 into which the head 8 of the femur 6 is received. As can be seen, the hip joint 10 is a ball and socket joint that provides multiple degrees of movement between the individual's legs and the pelvis to facilitate a variety of activities such as walking and running.

Cartilage 5, 7 lines the acetabulum 4 and head 8 of the femur respectively to provide a cushioning function to the joint 10 and to prevent the bones from rubbing together. To ensure that the head 8 of the femur is maintained in a close and stable position within the acetabulum 4, ligaments 3 are provided around and inside the joint 10. Muscles (not shown) which surround the hip joint 10 provide further stability to the hip joint 10.

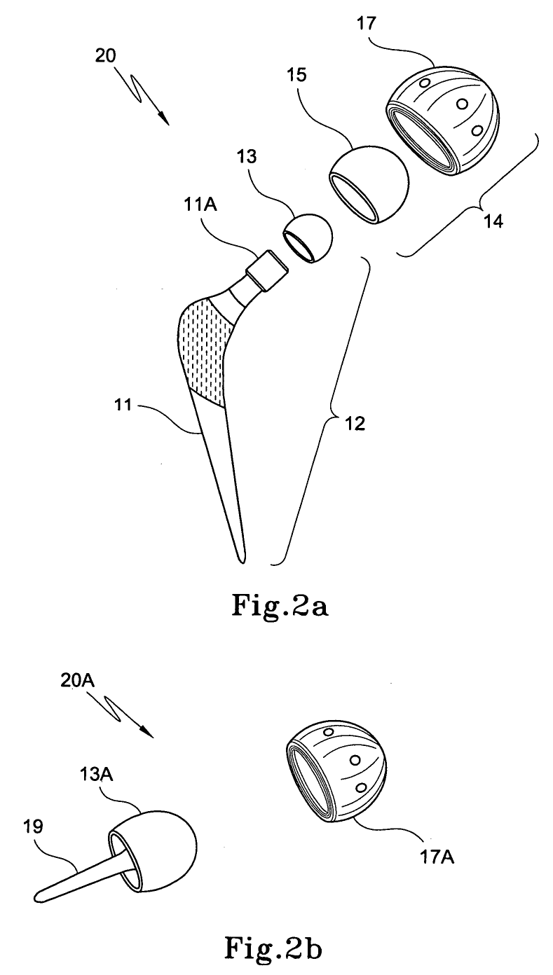

Conditions such as osteoarthritis may cause a deterioration and/or disintegration of the smooth cartilage surfaces 5 and 7 which in turn can lead to pain and restricted motion of the joint 10. This typically occurs as a gradual onset of worsening hip pain and decreased mobility in the joint 10 which makes normal walking and ascending and descending of stairs a progressively harder task. Should the condition worsen considerably, a total hip prosthesis 20 may be necessary, as is shown in FIG. 2A.

The prosthesis 20 generally comprises two portions, a femoral portion 12 and an acetabular portion 14. The femoral portion 12 comprises a metal step 11 which is configured to be placed into a marrow cavity formed in the femoral bone 6. The size and shape of the cavity being such that the stem 11 is tightly received therein and maintained in position. In this regard, bone cement may be applied to, assist in securing the stern 11 in position, or the stem 11 may have a surface texture which, over time, will allow the stem 11 to become secured in position through osseointegration with the femur.

A head element 13 is secured to the neck 11a of the stem 11 to function as the damaged femoral head 8 of FIG. 1. The head 13 is in the form of a ball, such as a ceramic or metal ball, which is sized in accordance with the individual's anatomical requirements.

The acetabular portion 14 generally comprises an insert 15 and a cup 17. The insert 15 is configured to be received within the cup 17 such that it is retained in position therein. The insert 15 is made from a ceramic or metal material and is shaped to receive the head 13 of the femoral portion when in position. The cup 17 is implanted into the acetabulum 4 of the pelvis 2. To facilitate implantation, the acetabulum 4 is drilled and prepared to create a recess whereby the cup 17 is securely fitted into the acetabulum 4. The cup 17 may be cemented in position within the acetabulum 4, or may be positioned through tightness of fit and/or screws, whereafter osseointegration may occur.

As is shown in FIG. 3, in this arrangement, the cup 17 and insert 15 articulates with the head element 13 to perform a ball-and-socket joint that replicates the natural hip joint 10 as shown in FIG. 1. As the cup 17 is seated in the hollowed acetabulum 4 of the pelvis 2 and the stem 11 is firmly secured in the marrow cavity of the femur 6, the forces produced by the body weight of the individual may pass from the pelvis 2 through the prosthesis 20 and into the femur 6. Such an arrangement provides even and appropriate body weight distribution on all parts of the skeletal structure of the individual.

As discussed previously, an alternative to total hip replacement is a procedure known as hip resurfacing, which unlike the procedure discussed above in relation to FIG. 2A, does not require a prosthesis that replaces the head 8 of the femur 6. A prosthesis 20a suitable for use in such a procedure is shown in FIG. 2B, whereby the head 8 of the femur 6 is substantially preserved and reshaped. In this regard, following reshaping of the head 8 of the femur 6 the resurfaced bone is capped with a head element 13a. The head element 13a comprises a stem portion 19 which is received by the femur 6 to secure the head element 13a in position. A cup or shell 17a, similar to that as discussed above in relation to the total hip prosthesis 20, is then implanted in the acetabulum 4 of the recipient's pelvis 2 to receive the head element 13a. An insert 15 may also be employed to fit within the shell 17a, as discussed above. The prosthesis 20a operates in a similar manner to that described above in relation to FIG. 3.

As will be appreciated, during use, such as during walking/running and various bending actions, the components of the prosthesis 20 experience a wide variety of forces in order to perform their function. Due to the wide variety of physical characteristics of individuals as well as the wide variety of surgical techniques employed to implant the prosthesis in the hip joint, forces exerted on the components may vary on an individual basis.

### Experimental Analysis

The present applicant conducted a review of 17 recipients of ceramic-on-ceramic hip prosthesis who had reported instances of an audible squeak resulting from their prosthesis. In order to investigate this occurrence further, orientation of the acetabular portion 14 of the prostheses was compared for each of the 17 squeaking prosthesis as well as for 17 matched recipients having prostheses with no reported instances of squeaking. Tests found that 94% of the non-squeaking implanted prosthesis were orientated in an ideal range of 25°+/−10° anteversion and 45°+/−10° inclination but only 35% of the squeaking implanted prostheses were in this range (p=0.0003). These results demonstrate the importance of acetabular portion orientation as one factor in the phenomenon of squeaking.

Of the 17 cases reporting squeaking prosthesis, eight of these exhibited squeaking during a bending movement, four exhibited squeaking during walking, whilst the remaining five exhibited squeaking of the prosthesis after prolonged periods of walking. Generally, it was found that prostheses that squeaked with walking had acetabular components that were more anteverted (40°) than prostheses that squeaked with bending (19°) (p=0.001) or following prolonged walking (18°) (p=0.020).

Generally, the prostheses started squeaking after an average of 14 months following implantation. The individuals were found to be younger, heavier and taller than patients with silent prostheses. Several of the individuals that reporting prosthesis squeaking underwent revision surgery to correct the phenomenon, thereby allowing the components of the squeaking prostheses to be retrieved and further analysed.

The analysis identified that common to all of the prostheses that exhibited squeaking was the evidence of edge loading and stripe wear between the insert 15 and the head 13 of the stem 11. Similarly, several of the prostheses that exhibited squeaking also showed evidence of impingement of the neck 11a of the prosthesis against the rim of the cup 17 of the acetabular portion.

Stripe wear is the term used to describe a long and narrow region of damage that is found on the head 13 of the stem as well as the inside of the insert 15 of a prosthesis. Stripe wear is the result of line contact between the head 13 and the edge of the insert 15. An example of what contributes to edge loading and stripe wear can be appreciated in considering a prosthesis recipient rising from a seated position. In such a physical action the recipient must firstly forcefully stretch the thigh that has been bent up, to an angle of at least 90°. When the thigh is bent in such a manner, the head 13 of the stem 11 is typically in contact with the back edge (rim) of the insert 15 of the cup 17. Upon stretching the thigh, the head 13 is initially against the back edge of the insert 15 before it begins to rotate with the individual's leg, thereby creating edge loading in the prosthesis.

To further investigate the audible squeak, sound recordings were further collected from over 30 patients with squeaking ceramic on ceramic hip prostheses and the sounds analysed by Fourier transformation to allow the major frequency components of the squeak to be determined.

The typical pattern was a harmonic series with a fundamental frequency between 400 Hz and 7500 Hz. Each patient has one or more characteristic fundamental frequencies that recurred with each squeak. Three patients recorded on two separate occasions had identical frequency signatures on both occasions.

In vitro studies were also carried out to determine the natural frequency of hip replacement components using an impulsive stimulus and an acoustic emission analysis. Titanium femoral stems and ceramic femoral heads both assembled and unassembled and modular ceramic/titanium acetabular components, which included testing the titanium shell and the respective ceramic inserts both assembled according to the manufacturers instructions and unassembled were tested.

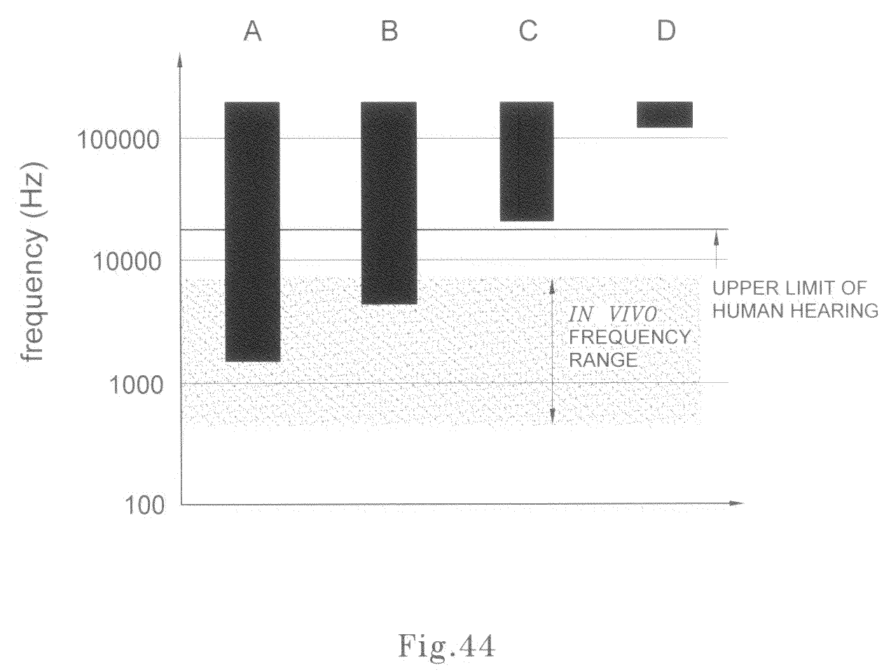

FIG. 44 shows the natural resonant frequencies of the components wherein A is the titanium stem; B is the titanium shell; C is the ceramic insert and D is the ceramic head.

No resonance was detected in the audible range in any of the modular ceramic/titanium acetabular components when they were correctly assembled and no resonance was detected in the audible range in any of the ceramic inserts or ceramic heads when tested unassembled.

Audible resonance was detected in all of the titanium shells when tested unassembled. The fundamental frequency of the titanium shell ranged from 4300 Hz to 9800 Hz with higher modes extending into higher frequencies. The thinner and larger shells tested had the lower frequency.

Of the ceramic inserts, when tested unassembled all were outside or at the limit of the audible range and only the thinnest of the larger diameter inserts had a fundamental frequency that was low enough to be detected by the equipment used (see FIG. 44).

The titanium femoral components had a minimum frequency around 1500 Hz and multiple natural frequencies in the human audible range between 2 kHz and 20 kHz.

Based upon the experimental data, it is considered that the audible squeak is a result of vibration between the components of the prosthesis during specific body movements causing the components to vibrate at the natural or harmonic frequencies, which happen to fall within an audible range.

In particular, it is considered that such vibrations are generated by the edge loading occurring between the head 13 of the stem 11 and the insert 15 of the cup 17 and/or impingement of the neck 11a against the rim 18 of the cup 17. The vibrations generated by the movement of the components of the prosthesis 20, namely the driving force, causes at least one of the components to resonate at its natural or resonant frequency. The frequency of this generated vibration will depend on the physical characteristics of the component(s) (including its mass, tension and stiffness) as well as the load between the surfaces and the magnitude of the forces present in the movement. The load and forces will change throughout the activity, such as bending to pick something up or walking up a flight of stairs, hence it is considered that the frequency of the vibrations being generated may change throughout the activity. Further, different components of the prosthesis 20 will have a different frequency response to the generated vibration.

Squeaking can be considered to be due to a dynamic response to an applied movement during gait. This is a case where there is a response to a stimulus and the head 13 bears against the acetabular cup insert 15. The stimulus is in the form of a rate of movement, friction, geometry and other factors. These lead to a load as a function of time. The response may vary due to variations in the stiffness, mass distribution and fixation of the acetabular cup 17 and liner 15 in addition to said variations in the femoral component.

Based upon the above described experimental analysis of the phenomena of squeaking reported in hip replacement prostheses, it is proposed that by altering the physical characteristics of components of the prosthesis, the natural or resonant frequencies of the components can be altered to occur outside the audible range of hearing of the human ear.

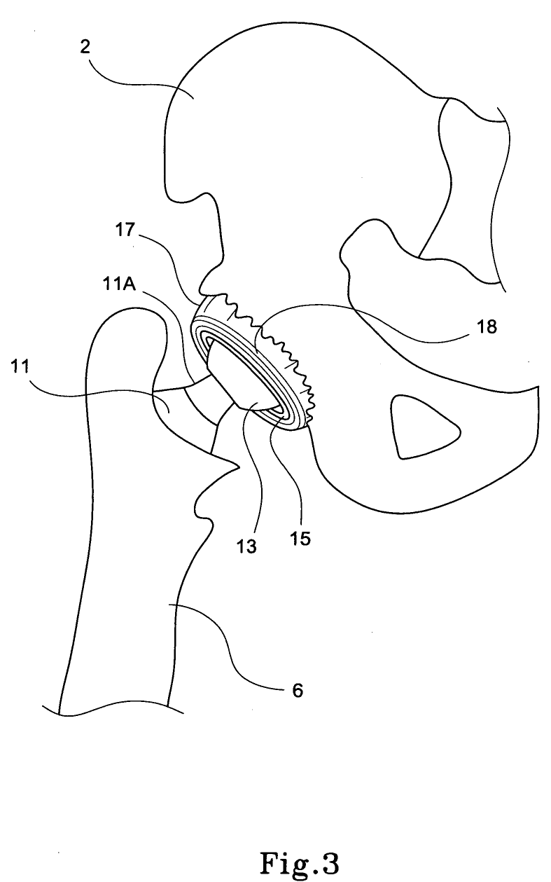

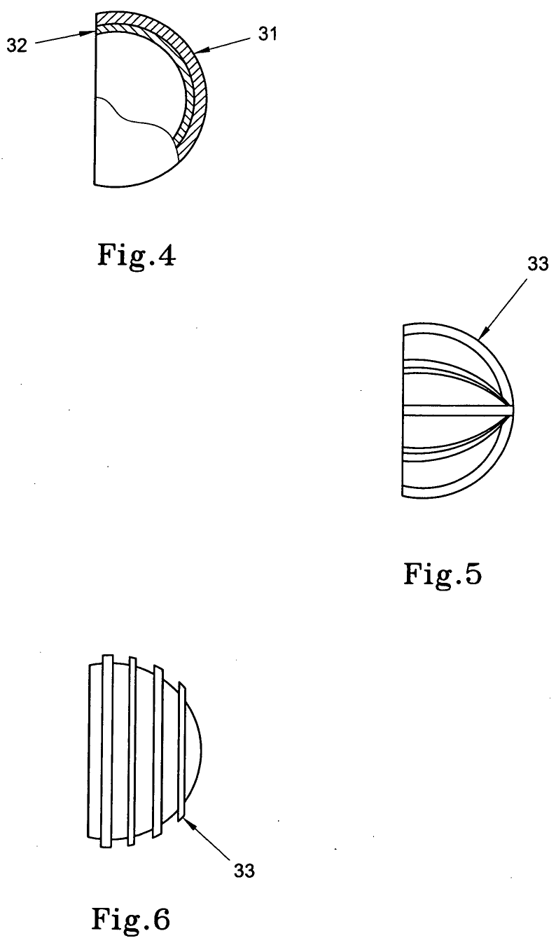

FIGS. 4 to 12 show a variety of ways in which the physical characteristics of the cup 17 of the prosthesis 20 can be altered to change the degree of response of the component and the natural or resonant frequency of the component such that any resonance emitted from the component due to vibrations generated therein are not audible to a human being.

In FIG. 4, the cup 17 is in the form of a composite, two layer structure made from different biocompatible materials, 31, 32. Each of the materials 31, 32 may have different stiffness properties to ensure that the resonant frequency of the cup falls outside the audible range. The number of layers and the thickness of each of the layers may be varied to dampen other frequencies.

Referring to FIGS. 11 and 12, a single damping material 36 may also be employed to alter the resonant frequency of the cup 17. As shown in FIG. 11 the damping material 36 forms an interface between inner 32 and outer 31 surfaces of the cup 17.

In FIG. 12, an adapter ring 37 is employed at the edge of the cup 17. The adapter ring 37 is in the form of a layered ring comprising damping material 38 between the inner and outer surfaces thereof, with the adapter ring 37 being located between the inner and outer surfaces of the cup 17. The thickness of the damping material 38 provided within the adapter ring 37 may vary depending on the frequencies that require damping or shifting out of the audible range.

FIGS. 5 and 6 show alternative embodiments for altering the geometric structure of the cup 17. In each of these embodiments ribs 33 are formed on the external surface of the cup 17. The ribs 33 may have various widths and thicknesses and the spacing and frequency of the ribs 33 on the external surface of the cup 17 may vary. As shown in FIG. 5, the ribs 33 may extend about the longitudinal axis of the cup 17, or may extend along the latitudes of the cup 17 as is shown in FIG. 6.

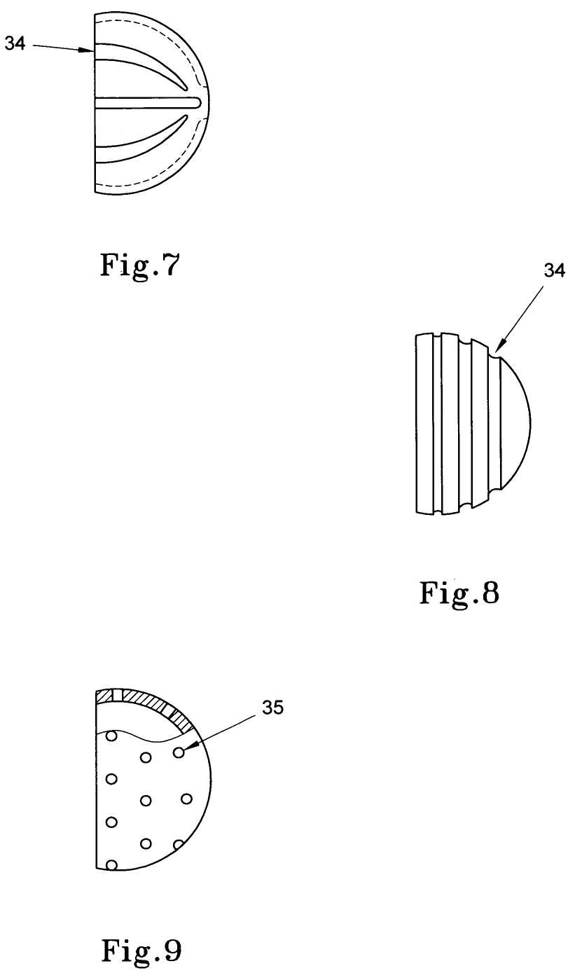

Referring to FIGS. 7 and 8, an alternative arrangement to alter the geometric structure of the cup 17 is to provide a number of grooves 34 in the external surface of the cup 17. As shown in FIG. 7, the grooves 34 may extend along different longitudinal directions and may extend through the surface of the cup 17. As shown in FIG. 8, the grooves 34 may also be arranged to extend along different latitudes of the surface of the cup 17, and the frequency, depth, spacing and number of grooves 34 employed may also vary.

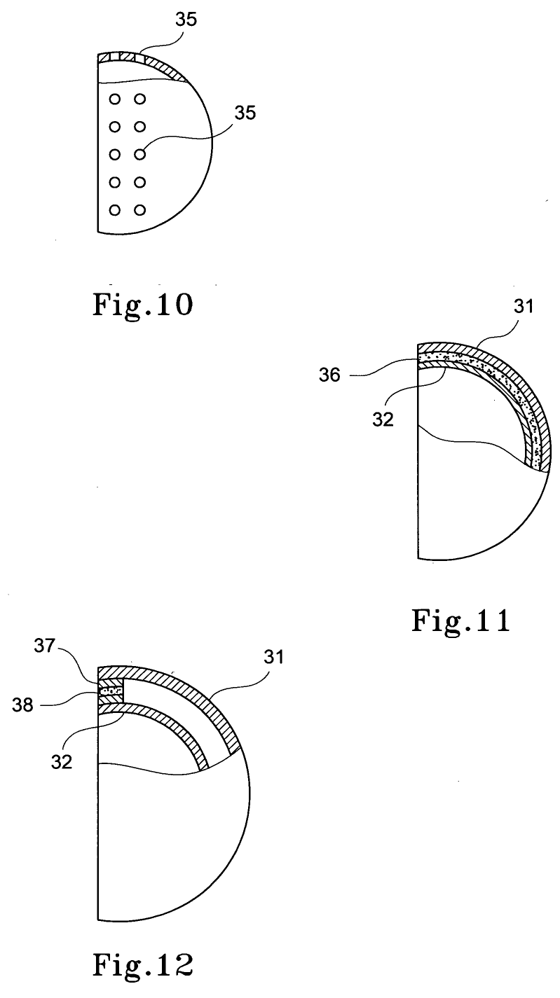

As shown in FIGS. 9 and 10, holes 35 extending partially or wholly through the surface of the cup 17 may also be employed to alter the geometric structure of the cup 17 and hence the resonant frequency of the component. As shown in FIG. 14, the holes 35 may be arranged to extend in different longitudinal directions along the surface of the cup 17, or may extend along different latitudes of the cup as shown in FIG. 10. Similarly the size, spacing and frequency of the holes 35 may vary depending upon the various design considerations.

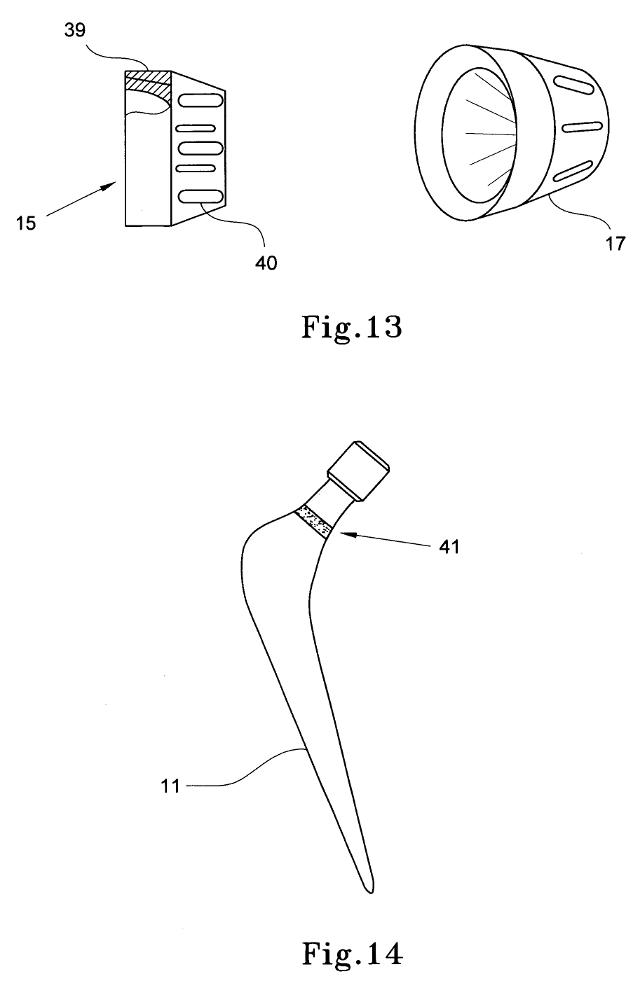

FIG. 13 provides an embodiment showing the manner in which the physical characteristics of the cup 17 and insert 15 of the prosthesis 20 can be altered to change the degree of response of the component and the natural or resonant frequency of the component such that any resonance emitted from the component due to vibrations generated therein, occur outside the audible range of human hearing. In this regard, a backing surface 39 is provided to the insert 15 such that when the insert 15 is positioned within the cup 17, this backing surface 39 forms a layer between the insert 15 and the cup 17. The geometric structure of the insert 15 may be altered by providing one or a number of slots or grooves 40 in the external backing 39 of the insert 15. As shown, the grooves or slots may extend along different longitudinal or latitude directions, and the frequency, depth, spacing and number of grooves employed may also vary.

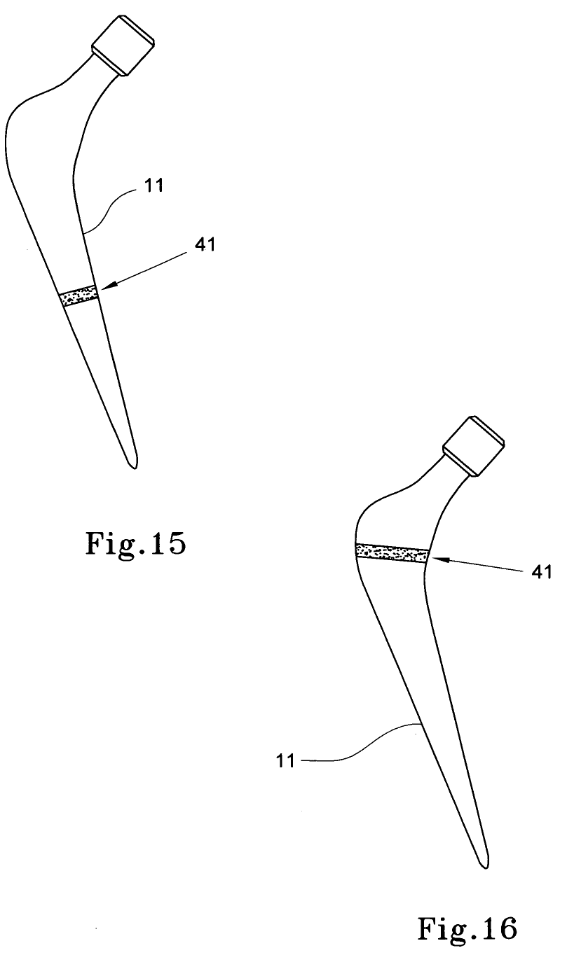

FIGS. 14 to 16 provide an embodiment showing the manner in which the physical characteristics of the femoral stem 11 of the prosthesis 20 can be altered to change the degree of response of the component and the natural or resonant frequency of the component such that any resonance emitted from the component due to vibrations generated therein, occur outside the audible range of human hearing. In these embodiments, a damping spacer 41 alters the geometric structure of the femoral stem 11 and thereby alters the resonant frequency. Referring to FIG. 14 the damping spacer 41 may be positioned along the neck 11a of the femoral stem 11 and may vary in material type and thickness. FIGS. 15 and 16 show the damping spacer 41 at two alternative locations along the stem 11. Similarly the size, spacing and frequency of the damping spacer 41 may vary depending upon the various design considerations.

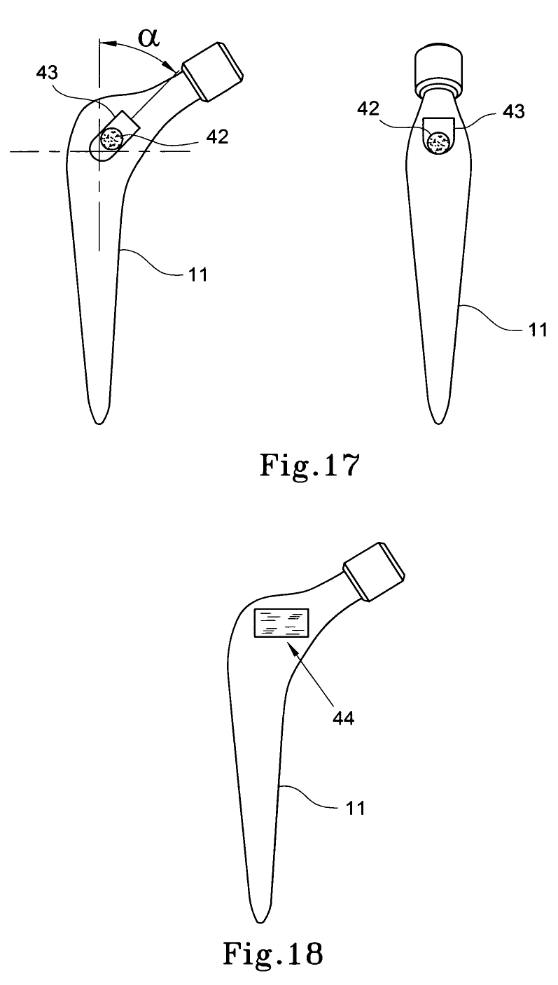

In FIGS. 17 and 18, a mass damper 42 alters the geometric structural response of the femoral stem 11 and thereby alters the resonant frequency. Referring to FIG. 17, the mass damper 42 is enclosed in the stem 11 and is allowed to move along a trough 43 and thereby adjusts itself to dampen a number of frequency modes. FIG. 18 employs a liquid mass damper 44 that is enclosed in a space within the stem 11 geometry. The liquid mass damper 44 moves out of phase to the vibrating frequency of the stem 11 and thereby dampens the frequency response. Similarly the size, spacing and frequency of the damping mass may vary depending upon the various design considerations. Further the damper 42/44 may be positioned anywhere along the length of the femoral stem ranging between the proximal and distal ends thereof.

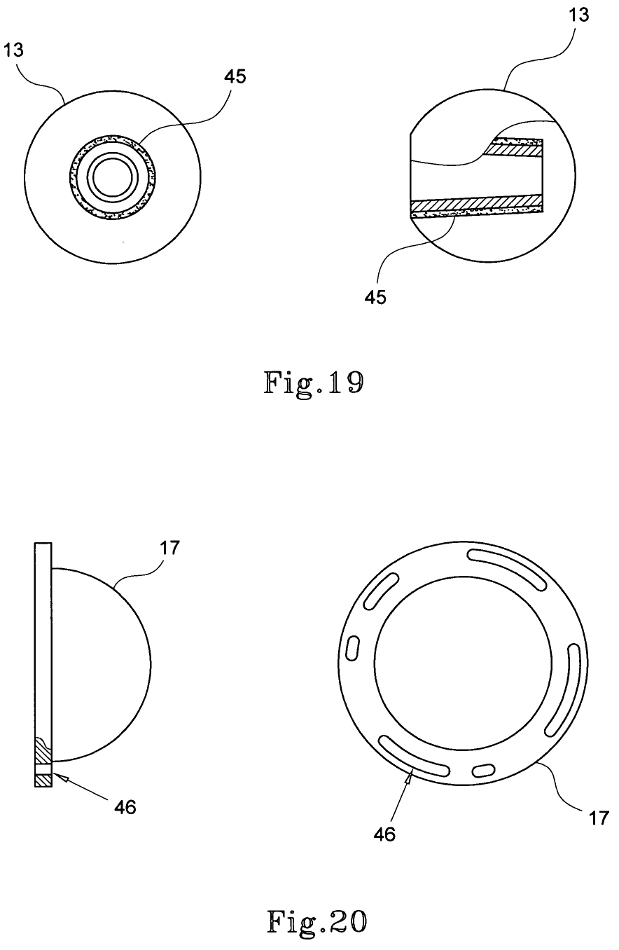

FIG. 19 provides an embodiment showing the manner in which the physical characteristics of the femoral head 13 of the prosthesis 20 can be altered to change the degree of response of the component and the natural or resonant frequency of the component such that any resonance emitted from the component due to vibrations generated therein, occur outside the audible range of human hearing. In this regard, a damping spacer material 45 is provided in the femoral head 13 of the prosthesis 20. The damping material 45 is placed between the taper interface of the femoral head 13 and stem 11a and the femoral head 13. The thickness and material type may vary depending on the design considerations.

As shown in FIG. 20, an embodiment showing the manner in which the physical characteristics of the acetabular cup 17 of the prosthesis 20 can be altered to change the degree of response of the component and the natural or resonant frequency of the component such that any resonance emitted from the component due to vibrations generated therein, occur outside the audible range of human hearing. In his embodiment, slots 46 have been employed to adjust the tuning of the acetabular cup 17. The slots 46 extend in a circumferential direction around the face of the acetabular cup 17. The size, depth spacing and frequency of the slots 46 may vary depending upon the various design considerations.

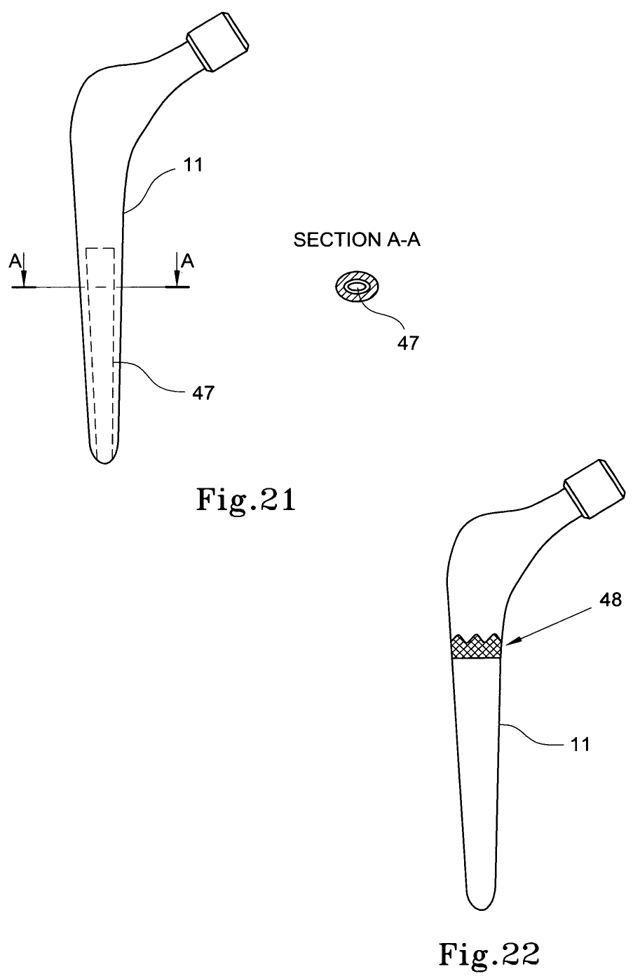

FIG. 21 shows yet another embodiment showing the manner in which the physical characteristics of the femoral stem 11 of the prosthesis 20 can be altered to change the degree of response of the component and the natural or resonant frequency of the component such that any resonance emitted from the component due to vibrations generated therein, occur outside the audible range of human hearing. In this embodiment, a portion of the femoral stem 11, in the form of an elliptical cylinder 47 is removed from the shaft of femoral stem 11. This length of the elliptical cylinder 47 removed from the stem 11 can vary depending upon the various design considerations.

As shown in FIG. 22, in another embodiment a torsional and axial damping spacer 48 can be employed to alter the geometric structure of the femoral stem 11 and thereby altering the resonant frequency. The damping spacer 48 may be positioned at any location or number of locations in the femoral stem 11 and may vary in material type, shape and thickness. Similarly the size, spacing and frequency of the damping spacer 48 or spacers may vary depending upon the various design considerations.

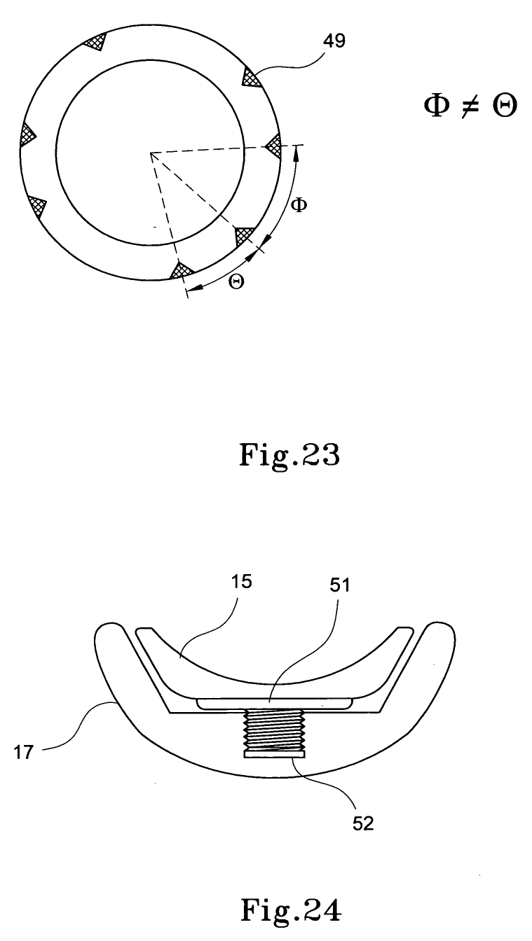

In FIG. 23, another embodiment is depicted showing the manner in which the physical characteristics of the acetabular cup 17 of the prosthesis 20 can be altered to change the degree of response of the component and the natural or resonant frequency of the component such that any resonance emitted from the component due to vibrations generated therein, occur outside the audible range of human hearing. In this embodiment, a notching sequence 49 has been employed around the perimeter of the acetabular cup 17. The angles between the notches 49 are not necessarily equal to generate a higher frequency mode that does not lie in the audible range. The notch geometry may vary to become grooves or slots depending on the various design considerations.

FIG. 24 shows yet another embodiment of the present invention wherein the acetabular portion of the prosthesis 20 is altered to alter the natural or resonant frequency of the component. In this embodiment, a damping device 51 is arranged between the insert 15 and the acetabular cup 17. To assist in positioning the acetabular cup 17, a recess or hole 52 is typically formed in the cup 17 for attachment with an inserter device (not shown). In this arrangement the device 51 may be secured to the inside of the cup 17 by engaging with the recess or hole 52, through a screw thread arrangement of the like. Alternatively, the device 51 may be freely positioned within the cup 17 or secured by a variety of alternative means.

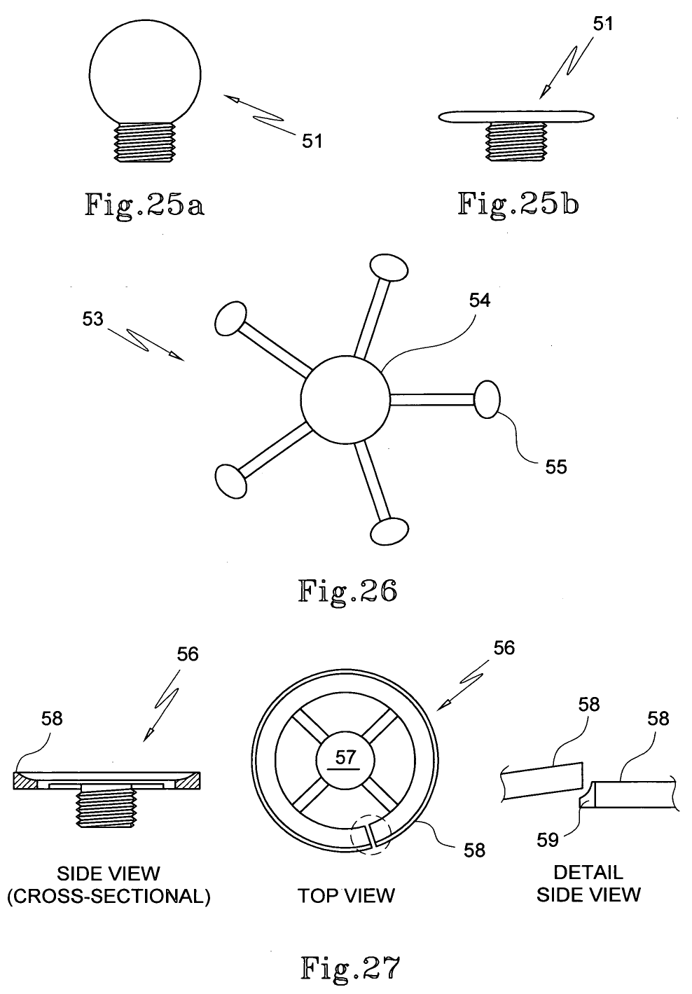

When positioned within the acetabular cup 17, the device 51 may be in a naturally expanded state as shown in FIG. 25A. When the insert 15 is positioned within the cup 17, the insert 15 compresses the device 51 into a compressed state as shown in FIG. 25B. In this regard, the insert 15 becomes seated and supported on the device 51 in its compressed state and as such any vibrations experienced by the cup 17 and insert 15, can be damped to prevent or substantially reduce squeaking of the prosthesis 20. It will be appreciated that the device 51 is made from a material that is compressible.

An alternative damping device 53 to that shown in FIG. 24 and FIGS. 25A and 25B, is shown in FIG. 26. In this embodiment, the device 53 has a main body 54 which is able to be attached to the recess or hole 52 of the cup 17 in the manner as discussed in relation to FIG. 24. A plurality of dampening attachments 55 extend from the main body 54 to contact and support the base of the insert 15 when the insert 15 is positioned within the cup 17. Similarly, in this arrangement, the insert 15 becomes seated and supported on the device 53 any vibrations experienced by the cup 17 and insert 15 can be damped to prevent or substantially reduce squeaking of the prosthesis 20.

FIG. 27 shows yet another alternative damping device 56, to be used in a manner as discussed in relation to FIGS. 24 to 26. In this arrangement, the device 56 comprises a main body portion 57 which is shaped to be attached to the recess or hole 53 formed in the cup 17, as previously discussed. A washer 58 is attached to the main body portion 57 of the device 56 which is provided to receive and contact the insert 15 when the insert 15 is inserted into the cup 17. A split 59 is provided in the washer to facilitate damping of any vibrations present in the prosthesis 20 such that when the insert 15 becomes seated and supported on the device 56, any vibrations experienced by the cup 17 and insert 15 can be damped to prevent or substantially reduce squeaking of the prosthesis 20.

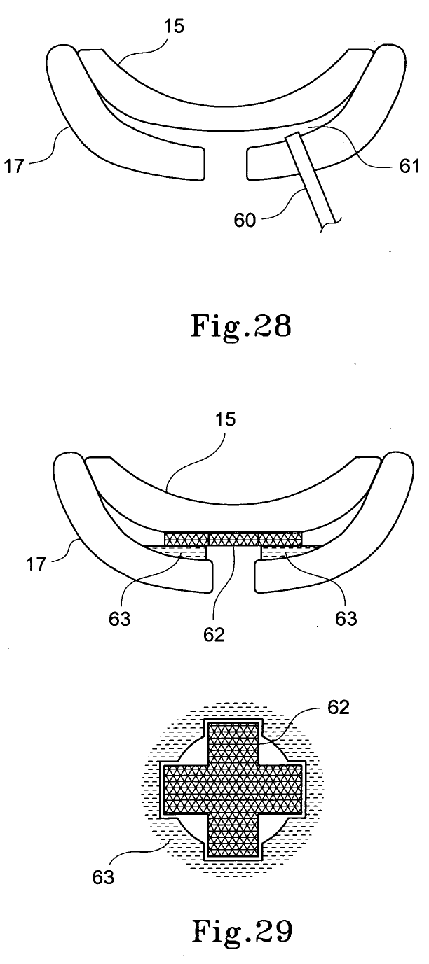

FIG. 28 shows yet another embodiment of the present invention wherein the acetabular portion of the prosthesis 20 is altered to alter the natural or resonant frequency of the component. In this arrangement, when the insert 15 is positioned within the cup 17, an instrument 60 is provided to penetrate the cup 17, through an existing hole in the cup 17 or through a hole made by the instrument 60 or another dedicated instrument, to access the interior space 61 between the cup 17 and the insert 15. The instrument 60 delivers damping material (not shown) in the form of a grout, putty or cement, such as polymethylmethacrylate, or a deformable material such as polyethylene or rubber, or any other form of a mechanical device that fills, or substantially fills the space 61. The filling of the space 61 with such a damping material acts to ensure that any vibrations experienced by the cup 17 and insert 15 can be damped to prevent or substantially reduce squeaking of the prosthesis 20.

FIG. 29 shows yet another embodiment of the present invention wherein the acetabular portion of the prosthesis 20 is altered to alter the natural or resonant frequency of the component. In this arrangement an attachment 62 is secured to an outer surface of the insert 15, which interacts with the cup 17, or a corresponding attachment 63 applied to the inner surface of the cup 17. The attachments 62, 63 alter the manner in which the cup 17 and insert 15 interact to stiffen the acetabular component thereby altering the resonant frequency response of the cup and insert component. Such an arrangement also acts to prevent or at least substantially reduce rotational movement between the insert 15 and the cup 17 thereby acting to dampen torsional vibration modes within the prosthesis 20. It will be appreciated that in the embodiment as shown, either or both of the attachments 62, 63 may be employed as necessary.

The above examples look at altering properties of the prosthesis to modify the resonance of individual components of the assembly. A further embodiment of the invention considers the interaction between components and in particular the cup 17 and the insert 15.

In particular, resonance of the cup 17 in an audible range may be prevented by locking the insert 15 and the cup 17 together under all loading pressures and particularly under loading of the femoral head on the edge of the insert such that the cup is not free to resonate at its audible natural frequency ie the insert and the cup act as a composite structure with a different resonant frequency to that of the cup alone.

The insert 15 and the cup 17 may be locked together using mechanical details or by altering other variables including friction, taper angle of the insert 15 and the stiffness of the cup 17. By providing a good locking mechanism between the cup 17 and the insert 15, the likelihood of the insert 15 disengaging from the cup during normal or abnormal gait activities is prevented or at least substantially reduced. Such disengagement between the cup 17 and insert 15 can change the load characteristics of the prosthesis thereby producing vibrations within the prosthesis 20 which contribute to the emission of an audible squeak from the prosthesis.

In FIGS. 30 to 36, the locking mechanism is achieved by providing a stabilising ring 70 adapted to be attached to the cup 17 to restrict movement of the insert 15 within the cup 17.

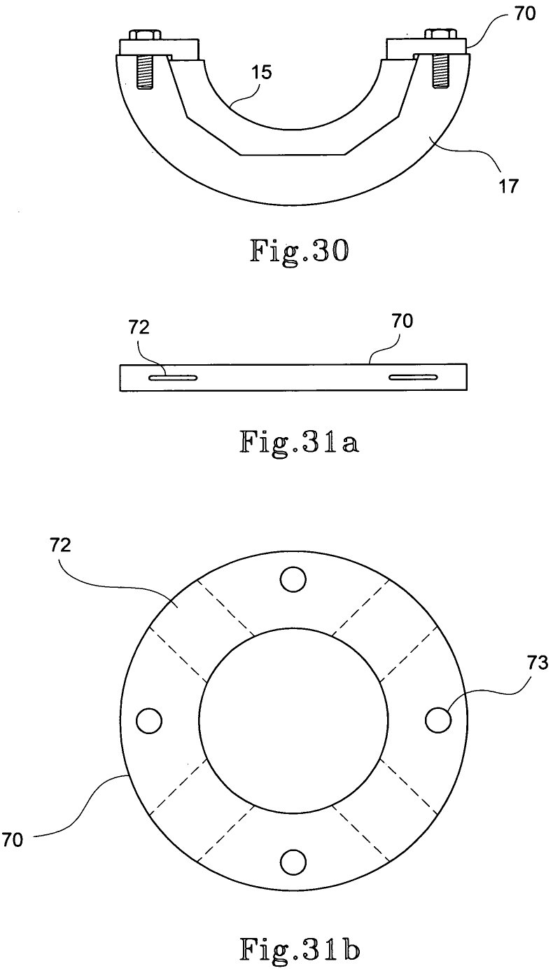

As shown in FIGS. 31A and 31B, in one form the stabilising ring 70 may be in the form of a substantially flat ring element having stabilising slots 72 formed therein for compliance. A plurality of holes 73 may also be provided to receive one or more screws or the like for retaining the ring 70 in position against the upper rim of the cup 17, as shown in FIG. 30.

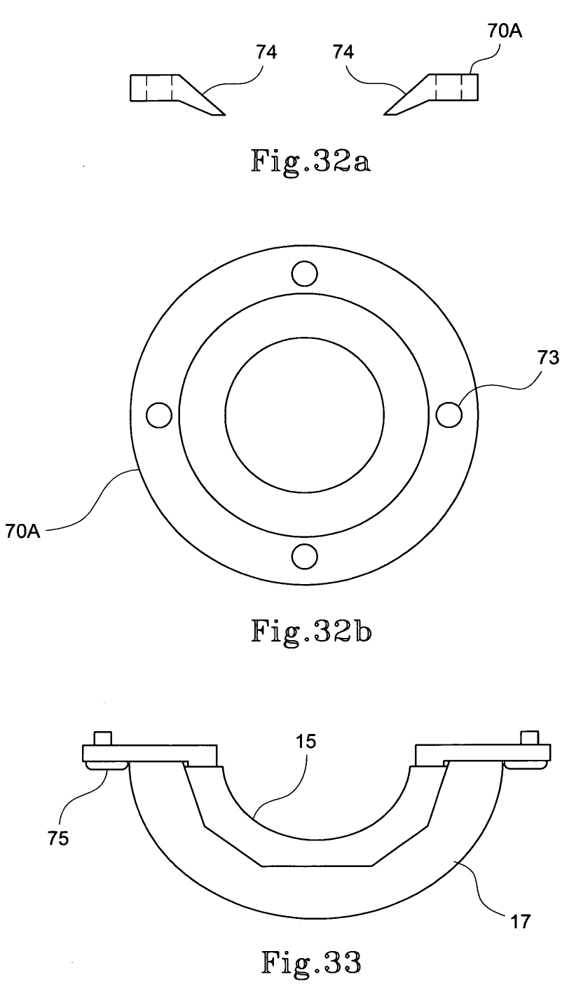

An alternative embodiment of the stabilising ring 70a is shown in FIGS. 32A and 32B. In this embodiment, the ring 70′a has a downwardly projecting foot portion 74 which extends into the cup 17 when the ring 70a is secured to the rim of the cup 17. In this arrangement the foot portion 74 contacts the insert 15 to provide a stabilising force against the insert 15 to maintain it in position with respect to the cup 17.

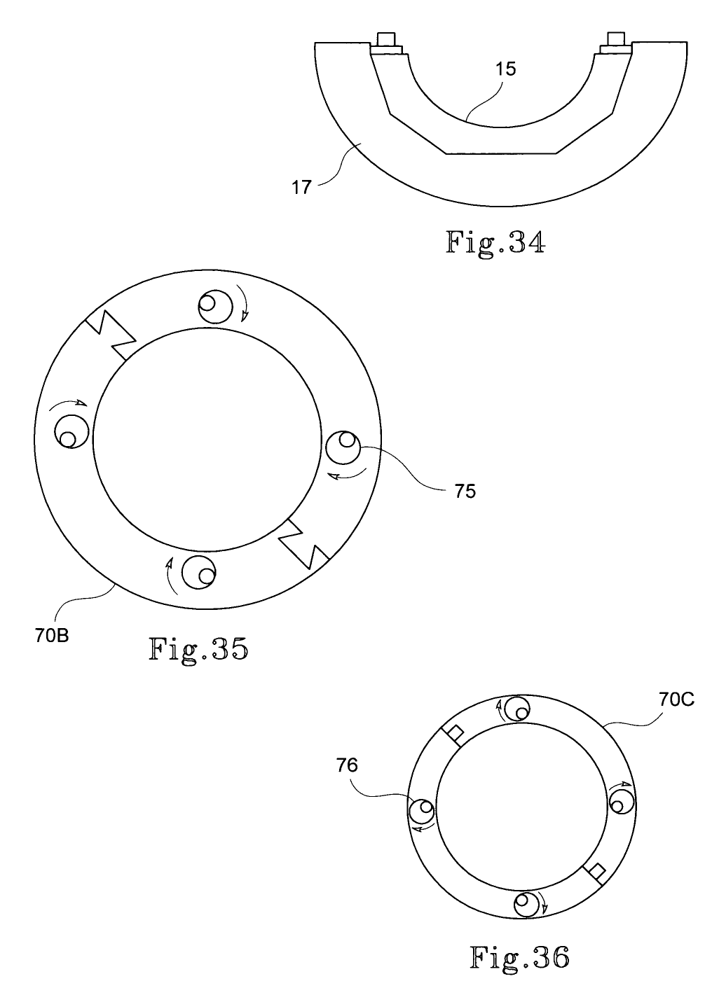

Another embodiment of the stabilising ring 70b is shown in FIG. 35. In this embodiment, the ring 70b comprises one or more locking lugs 75 which are rotated in the direction of the arrow to lock against the outside of the cup 17, as shown in FIG. 38. In this regard, the ring 70b is placed over the rim of the cup 17 and insert 15 such that the lugs 75 are rotated to engage the external surface of the cup 17 adjacent the rim of the cup, to limit any unwanted movement of the insert 15 with respect to the cup 17. FIG. 36 shows yet another embodiment of the ring 70c, whereby the ring 70c is adapted to fit within the cup 17, atop the insert 15 to restrict unwanted movement of the insert 15 with respect to the cup 17. In this arrangement, locking lugs 76 are employed to engage with the inside of the cup 17, as shown in FIG. 34, to secure the ring 70c in position.

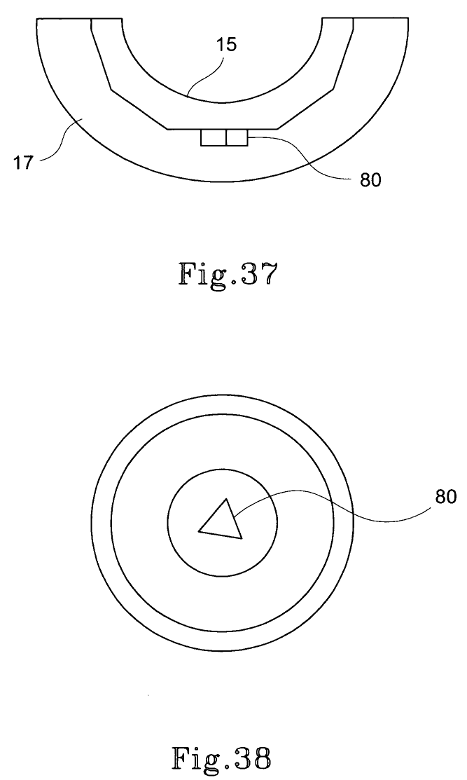

FIGS. 37 and 38 depict yet another embodiment for restricting movement of the insert 15 with respect to the cup 17 to reduce the potential of the prosthesis emitting an audible squeak. In this embodiment, a locking post 80 may be affixed to the base of the insert 15 to be received in a shaped recess provided in the interior of the cup 17. FIG. 38 represents the underside of the insert 15 which shows the post 80 having a substantially triangular shape, which is received in a substantially triangular hole formed in the interior of the cup 17, to restrict unwanted movement between the components. It will be appreciated that the actual geometry of the post 80 may vary. Further, the post 80 may be part of the insert 15, or fixed to the insert 15.

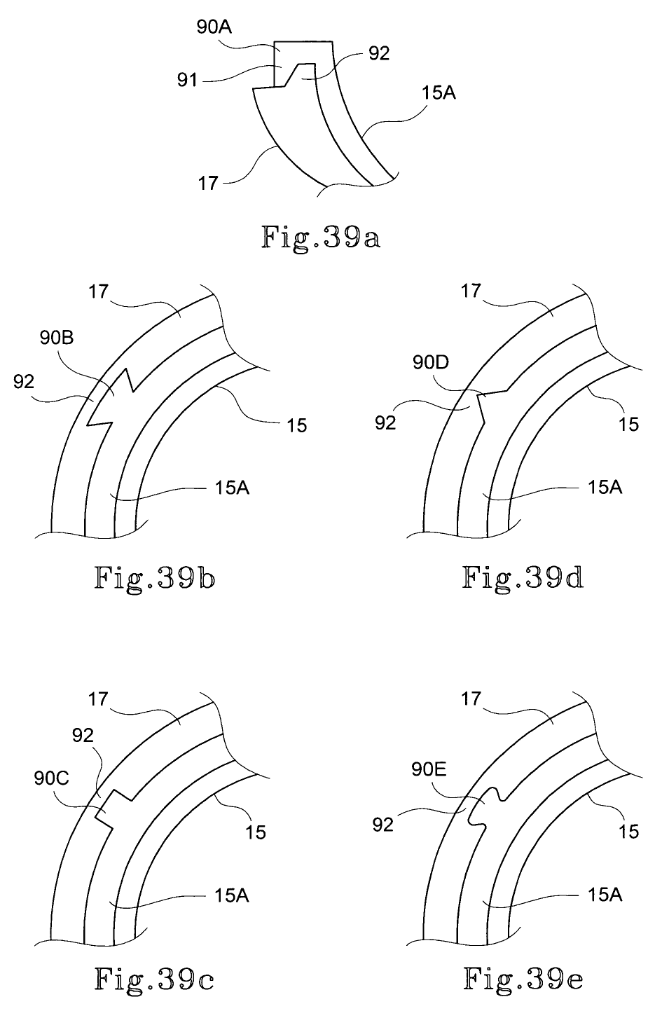

The embodiments of the invention depicted in FIGS. 39a to 39e provide a further locking mechanism to hold the insert 15 and the cup in locking engagement under various loads. The insert 15 includes a plurality of mechanical details 90 to engage a receiving portion 92 of the cup 17. The mechanical details 90 may be formed on the insert 15 itself or, as depicted, may be part of an intermediate component 15a that is adapted to fit between the insert 15 and the cup 17. Intermediate component 15a includes locking details depicted as 90a, 90b, 90c, 90d and 90e in FIGS. 39a) to e) respectively. In FIG. 39a, the detail comprises a hook 91 that engages with recessed receiving portion 92 of the cup 17.

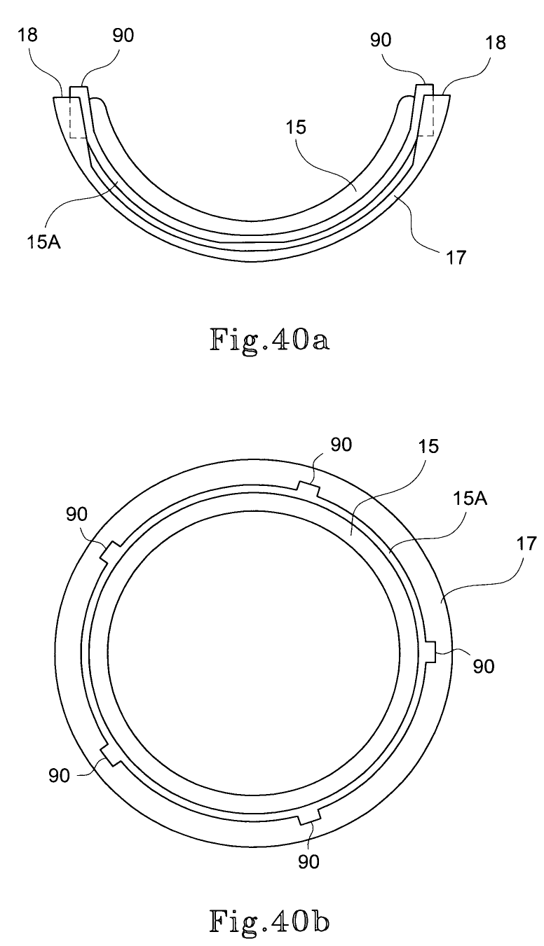

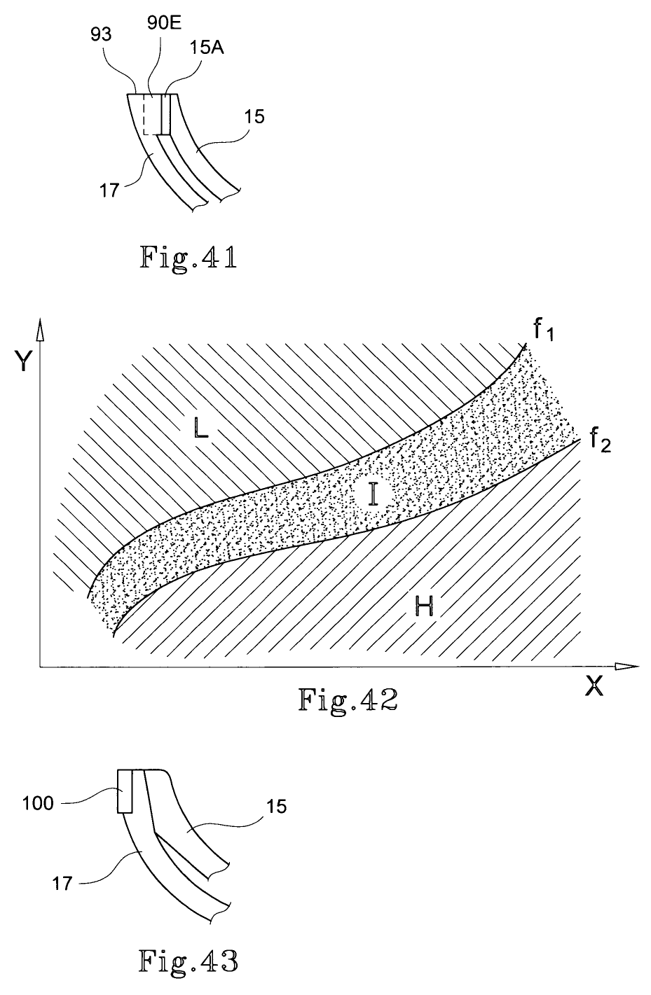

The plan views depicted in FIGS. 39b) to 39e) show the different shapes of mechanical detail that may be used to lock the insert 15 to the cup 17. The intermediate member 15a may extend above rim 18 of cup 17 as shown in FIG. 40a. Alternatively, the intermediate member 15a may be flush with the rim 93 of the cup 17 as shown in FIG. 41. In use, the insert 15 and intermediate member 15a are positioned such that the mechanical details 90 align with the corresponding recessed portions of the cup to enable easy insertion of insert 15. The insert is then fitted into the cup. In this regard, and in the case of most prior art prostheses that have an insert and a cup, the interior of the cup 17 is provided with a taper complementary to the taper on the exterior of the insert 15. The engagement of the complementary tapers is usually sufficient to retain the insert 15 within the shell or cup 17 during axial compression. However, and as discussed earlier, the taper may not be sufficient to lock the insert within the cup during edge loading or impingement, both of which form part of the normal loading spectrum of the prosthesis. The engagement of the mechanical details 90 with the recessed portions of the cup provide a further locking mechanism that prevents sliding or tipping of the insert 15 during edge loading and impingement. The insert 15 is, therefore, far less likely to disengage from the cup 17 and thus the likelihood of squeaking resulting from the resonance of the cup when separated from the insert greatly reduced, if not abolished.

The angle of the complementary tapers of the insert 15 and the cup 17 and the stiffness of the cup 17 can increase the risk of squeaking of the prosthesis during use. As shown in FIG. 42, there are two functions f1 and f2 that define this interrelationship. The area marked L denotes a low risk of squeaking, I an intermediate risk of squeaking and H a high risk of squeaking. As the cup 17 stiffness (axis y) increases and the taper angle (axis x) decreases the propensity to squeak will decrease. Conversely as the cup stiffness decreases and the taper angle increases the propensity to squeak will increase. The function f1 defines the combination of cup stiffness and taper angles which represent a low risk of squeaking. The function f2 defines the combination of cup stiffness and taper angles which represent a high risk of squeaking. f1 and f2 are functions of taper angle and cup stiffness but f1 and f2 will also have dependencies on other parameters including cup sizes, cup materials and cup geometries. This principle and a similar interrelationship will also apply for locking mechanisms other than a taper, where there is a potential for non-composite action.

To increase the stiffness of the cup 17 and thus improve the locking of the insert 15 and the cup 17, the cup 17 may include a stiffening ring 100. The stiffening ring 100 is made of a stiffer material than the material of the cup 17, that is, it has a higher Young's modulus than the cup 17. The stiffening ring increases both the hoop and the bending stiffness of the cup thereby reducing the propensity of the insert to disengage from the cup.

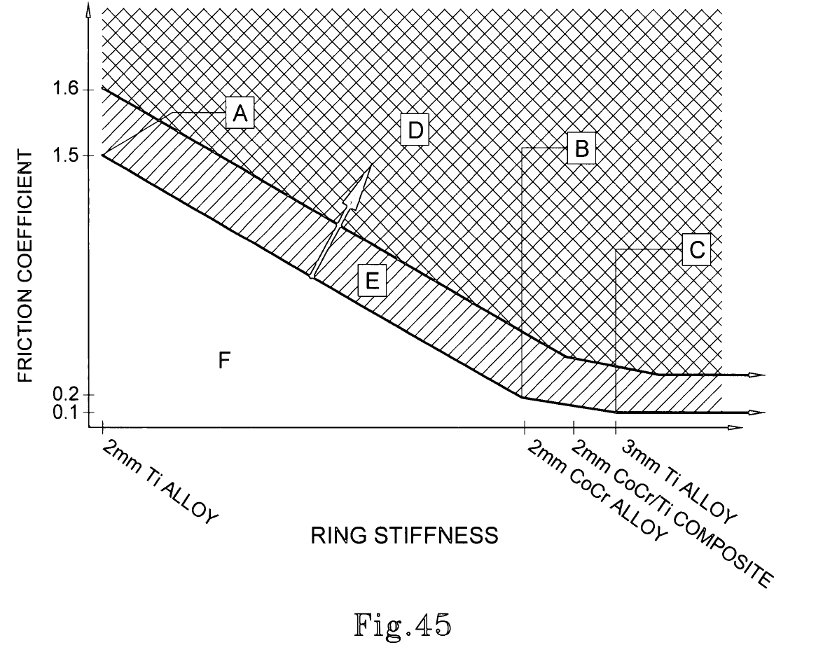

Another important variable in providing a non-squeaking prosthesis is the friction between the insert 15 and the cup 17. FIG. 45 presents the relationship between friction coefficient and ring stiffness of the cup 17. As may be seen, if the friction coefficient is low, a high ring stiffness would be required to ensure a composite action between the insert 15 and the cup 17.

Item A denotes a friction coefficient of 1.5 and a cup stiffness for a 2 mm thick Titanium alloy. Item B denotes a function coefficient of 0.2 mm and a cup stiffness for a 2 mm thick Cobalt Chrome alloy. Item C denotes a friction coefficient of 0.1 and a cup stiffness for a 3 mm Titanium alloy. The area of the graph marked D is the region wherein the insert and the cup will act as a composite member under all clinically relevant loads. Region E is a lower risk region for squeaking and region F is a high risk region for squeaking.

The particular examples provided at items A, B and C demonstrate the relationship between stiffness and friction coefficient. However, there are many possible combinations and distributions of material that can result in similar ring stiffness. The following equations allow the relative stiffness of these combinations to be compared and encompass all combinations of variables as outlined herein.

For a homogenous material the ring stiffness is given by the formula:

$$\frac{{Et}^{3}}{12} ⇔ stiffness per millimetre run$$

where E is the Young's modulus of the material
t is the thickness
For a composite material with centroid of each component being xᵢ giving a combined centroid of x̅ given by the formula.

$$\overline{x} = \frac{\sum {E}_{i} {x}_{i} {t}_{i}}{\sum {x}_{i} {t}_{i}}$$

The ring stiffness is given by the formula:

$$y ( \frac{ \sum {t}_{i}^{3} {E}_{i}}{12} + \sum {( {x}_{i} - \overline{x} )}^{2} {t}_{i} {E}_{i} )$$

Where y is the mobilised length of the ring.

An optimal combination of ring stiffness and friction coefficient and/or taper angle will provide a composite structure wherein movement of the insert 15 and the cup 17 is minimised, and preferably to less than 40 microns.

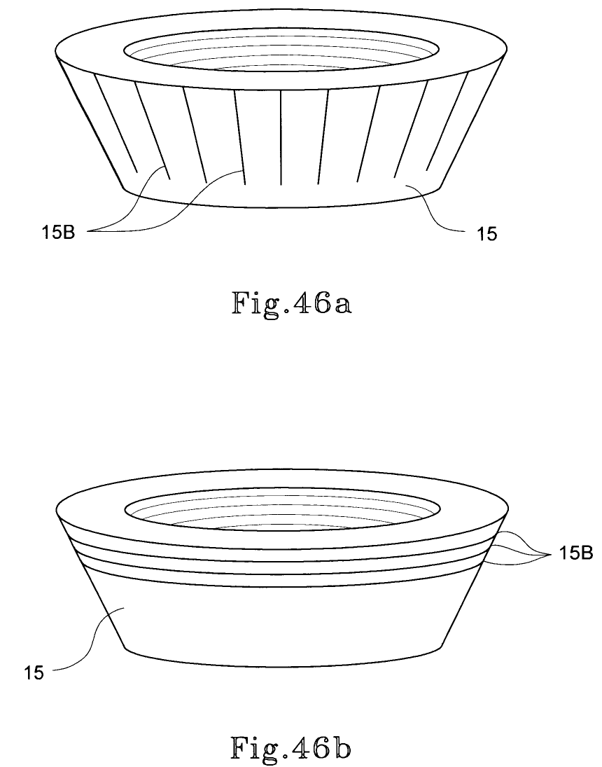

FIGS. 46a and 46b depict an insert member 15 that includes surface details 15b to modify the friction properties of the insert 15. Other examples include roughening the outer surface of the insert 15.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An implantable joint prosthesis comprising:
a first component attachable to a first bone of a recipient; and
a second component attachable to a second bone of a recipient,
wherein said first and second components are arranged to facilitate relative movement between said first and second bone of the recipient, and at least one of said first and/or second components comprises at least one modifying means, said modifying means modifying the first and/or second component such that a dynamic response of at least a part of the first and/or second component to a stimulus is modified.

2. The implantable joint prosthesis of claim 1 wherein the dynamic response comprises a resonant frequency of at least a part of the first and/or second components.

3. The implantable joint prosthesis of claim 1 wherein a magnitude of the dynamic response is modified.

4. The implantable joint prosthesis of claim 3 wherein any noise resulting from a resonance of said at least a part of the first and/or second component is reduced to a level that is not audible to a human.

5. The implantable joint prosthesis of claim 1 wherein a frequency of the dynamic response is modified.

6. The implantable joint prosthesis of claim 5 wherein the frequency of the dynamic response is modified to greater than 7 KHz.

7. The implantable joint prosthesis of claim 5 wherein the frequency of the dynamic response is modified to greater than 10 KHz.

8. The implantable joint prosthesis of claim 5 wherein the frequency of the dynamic response is modified to a range of 10 KHz to 20 KHz.

9. The implantable joint prosthesis of claim 5 wherein the frequency of the dynamic response is modified to a frequency greater than 20 KHz.

10. The implantable joint prosthesis of claim 1 wherein both a magnitude a particular frequency of the dynamic response are modified.

11. The implantable joint prosthesis of claim 1, wherein the implantable joint prosthesis is an an implantable hip prosthesis, wherein said first component comprises a femoral component and said second component comprises an acetabular component having an acetabular cup shaped to receive an insert therein.

12. The implantable joint prosthesis of claim 11 wherein the modifying means comprises one or more shape modifying members of the acetabular component.

13. The implantable joint prosthesis of claim 12 wherein the shape modifying members comprise one or more ribs or struts wherein said ribs or struts extend outwardly from an outer surface of the acetabular component.

14. The implantable joint prosthesis of claim 11 wherein the modifying means comprises at least one stiffness modifying member of the acetabular component.

15. The implantable joint prosthesis of claim 14 wherein the stiffness modifying member comprises one or more ribs or struts wherein said ribs or struts extend outwardly from an outer surface of the acetabular component.

16. The implantable joint prosthesis of claim 14 wherein the stiffness modifying member comprises a ring member that is stiffer than the acetabular cup.

17. The implantable joint prosthesis of claim 16 wherein the ring member is made from the same material as the material of the acetabular cup and includes stiffening features to increase the stiffness of the ring member.

18. The implantable joint prosthesis of claim 17 wherein the stiffening features comprise one or more extension members that extend from the ring member.

19. The implantable joint prosthesis of claim 16 wherein the ring member is made from a different material to the material of the acetabular cup.

20. The implantable joint prosthesis of claim 16 wherein the ring member extends substantially circumferentially around an outer surface of the acetabular cup.

21. The implantable joint prosthesis of claim 11 wherein the modifying means comprises a locking mechanism.

22. The implantable joint prosthesis of claim 21 wherein the locking mechanism comprises a locking member positioned on the insert of the acetabular component and a complementary receiving member on the acetabular cup.

23. An acetabular component of an implantable hip prosthesis, said acetabular component having a main axis and a cup member shaped to receive an insert member substantially therein, wherein the insert member and the cup member are coupled together by a primary locking mechanism, said primary locking mechanism retaining the insert member and the cup member in coupling engagement when said acetabular component is subjected to a load substantially along said main axis; and
the acetabular component includes a secondary locking mechanism to couple together the insert member and the cup member.

24. The acetabular component of claim 23 wherein said secondary locking mechanism retains the insert member and the cup member in coupling engagement when said acetabular component is subjected to a load that deviates from said main axis.

25. The acetabular component of claim 24 wherein the secondary locking mechanism retains the insert member and the cup member in locking engagement when the load applied to the acetabular component is at an angle to the main axis.

26. The acetabular component of claim 25 wherein the angle of the load is between 1° and 90° relative to the main axis.

27. The acetabular component of claim 23 wherein the secondary locking mechanism modifies the dynamic response of at least a part of the acetabular component to a stimulus.

28. The acetabular component of claim 27 wherein the dynamic response comprises the resonant frequency of at least a part of the acetabular component.

29. The acetabular component of claim 23 wherein the secondary locking mechanism comprises a first locking member on the insert member and a second locking member on the cup member.

30. The acetabular component of claim 29 wherein the first locking member comprises an extension member of the insert and the second locking member comprises a recessed portion of the cup member and wherein the recessed portion is substantially configured to receive the extension member.

31. The acetabular component of claim 30 wherein the insert member further includes an intermediate member that extends around at least a portion of an outer surface of the insert member, and the intermediate member comprises said extension member.

32. The acetabular component of claim 23 wherein the secondary locking mechanism comprises at least one stiffness modifying member of the cup member.

33. The acetabular component of claim 32 wherein the stiffness modifying member comprises one or more ribs or struts on an outer surface of the cup member.

34. The acetabular component of claim 32 wherein said stiffness modifying member comprises a ring member stiffer than the cup member.

35. The acetabular component of claim 34 wherein the ring member includes stiffening members.

36. The acetabular component of claim 35 wherein the stiffening members include ribs or struts extending from the ring member

37. The acetabular component of claim 34 wherein the cup member is made of titanium alloy and the ring member is made of a different material to the material of the cup member including cobalt chrome alloy and stainless steel.

38. The acetabular component of claim 34 wherein the ring member extends substantially circumferentially around the entire circumference of the cup member.
